(19) United States
(12) Patent Application Publication
(10) Pub. No.: US 2012/0142634 A1
(43) Pub. Date: Jun. 7, 2012

(54) Method of Treating Cancer with a Combination of a Proteasome Inhibitor and Salubrinal

(75) Inventors: Denis M. Schewe, Westerroenfeld (DE); Julio A. Aguirre-Ghiso, Cortlandt Manor, NY (US)

(73) Assignee: MOUNT SINAI SCHOOL OF MEDICINE OF NEW YORK UNIVERSITY, New York, NY (US)

(21) Appl. No.: 13/128,448

(22) PCT Filed: Nov. 10, 2009
(86) PCT No.: PCT/US2009/063889

§ 371 Date: Feb. 21, 2012

Related U.S. Application Data
(60) Provisional application No. 61/113,208, filed on Nov. 10, 2008.

(51) Int. Cl. A61K 31/4706; A61P 35/00; A61K 31/69

(52) U.S. Cl. 514/64; 514/311

(57) ABSTRACT

The disclosure provides methods for treating or preventing a cancerous condition, such as multiple myeloma, by administering a therapeutically effective combination of a proteasome inhibitor and salubrinal.

### CROSS REFERENCE TO RELATED APPLICATIONS

This application claims the benefit of priority of U.S. Provisional Application No. 61/113,208, which was filed Nov. 10, 2008, which is incorporated herein by reference in its entirety.

### GOVERNMENT INTEREST

This invention was made with U.S. government support under grant no. CA109182, awarded by the National Cancer Institute of the National Institutes of Health. The U.S. government has certain rights in the invention.

### FIELD OF THE INVENTION

The claimed subject matter relates generally to the field of cancer therapy and, more particularly, to the treatment of such cancers as multiple myeloma, using a therapeutically effective combination of a proteasome inhibitor and salubrinal.

### BACKGROUND

Proteasome inhibitors induce multiple myeloma (MM) cell apoptosis by modulating several pathways including endoplasmic reticulum (ER) stress signaling. The use of prolonged Bortezomib therapy, however, has led to the development of drug resistance. Drug resistance has been linked to enhanced ER stress signaling and increased chaperone expression, which may be an adaptive capacity of MM cells.

Proteasome inhibitors induce an ER stress response in MM cells contributing to apoptosis. However, a primary function of ER stress signaling is also to adapt to exogenous stress and induce growth arrest and survival. This is achieved in part by reducing cyclin D1 levels and inducing protein folding and degradation genes that alleviate the damage caused by unfolded proteins.

Depending on the activation intensity and cellular context, eIF2α phosphorylation by upstream kinases like PERK or GCN2 can induce survival, growth arrest and/or apoptosis in response to ER stress. Eukaryotic Initiation Factor 2α signaling has been shown to be linked to the induction and maintenance of HEp3 head and neck squamous carcinoma cell dormancy and survival. Growth arrest (i. e., dormancy) is in part due to PERK-dependent phosphorylation of eIF2α, which leads to the downregulation of Cyclins D1/D3 and CDK4. Survival and resistance to chemotherapy, on the other hand, is due to induction of BiP, ATF6 activation and also eIF2α phosphorylation. In other cases, very intense eIF2α phosphorylation can activate apoptotic programs. The capacity of eIF2α signaling to decide cell fate in response to stress might be particularly important for MM patients treated with proteasome inhibitors because different levels of stress may impinge on the cells during this therapy.

For the foregoing reasons, it is apparent that a need persists in the art for anti-cancer therapies that effectively target cancerous cells while also minimizing the risk of, and preferably preventing, a recurrence of that cancer in an animal such as a mammal, e.g., a human.

### SUMMARY

The disclosure satisfies at least one of the aforementioned needs by providing for treating or preventing any of a variety of cancers using therapeutic peptides capable of proteasome inhibition in combination with a protein interfering with the functional interaction of GADD34 and eIF2α. An exemplary cancer or cancerous condition is multiple myeloma. Exemplary proteasome inhibitor peptides are Bortezomib (i.e., Velcade®) and MG 132. An exemplary protein interfering with the functional interaction of GADD34 and eIF2α is salubrinal.

One aspect of the disclosure is drawn to a method for treating cancer comprising administering a therapeutically effective combination of a proteasome inhibitor and a protein interfering with the functional interaction of GADD34 and eukaryotic Initiation Factor 2α, that is, a protein that interferes with FADD34-PP1c complex assembly salubrinal to an organism in need. In a related aspect, the disclosure provides a method for treating cancer comprising administering a therapeutically effective combination of a proteasome inhibitor and a protein that inhibits Grp78 function to an organism in need. In some embodiments of either of these aspects of the disclosure, the cancer or cancerous condition is multiple myeloma. The proteasome inhibitor may be selected from the group consisting of MG 132 and Velcade® in some embodiments. The protein that interferes with the functional interaction of GADD34 and eukaryotic Initiation Factor 2α may be salubrinal. In some embodiments, the organism in need is a human, a human pet, domesticated livestock or a zoo animal.

In a related aspect, the disclosure provides a method for preventing a cancerous condition comprising administering a prophylactically effective combination of a proteasome inhibitor and a protein that interferes with the functional interaction of GADD34 and eukaryotic Initiation Factor 2α to an organism in need. An aspect of the disclosure related to this method is A method for preventing cancer comprising administering a prophylactically effective combination of a proteasome inhibitor and a protein that inhibits Grp78 function to an organism in need. As with methods for treating a cancer or cancerous condition, some embodiments each of these methods of prevention involve multiple myeloma. Suitable proteasome inhibitors include, but are not limited to, MG 132 and Bortezomib (Velcade®). An exemplary protein that interferes with the functional interaction of GADD34 and eukaryotic Initiation Factor 2α is salubrinal. The disclosure contemplates methods of preventing a cancer or cancerous condition in a human, a human pet, domesticated livestock or a zoo animal.

Another aspect of the invention is a kit suitable for practicing any of the aforementioned methods. Such kits contain a proteasomal inhibitor and a protein that interferes with the functional interaction of GADD34 and eukaryotic Initiation Factor 2α, along with a protocol guiding administration of a therapeutically, or prophylactically, effective combination of the therapeutics. A related kit contains a proteasomal inhibitor and a protein that inhibits Grp78 function along with a protocol for administration of the therapeutics or prophylactics.

Other features and advantages of the present disclosure will be better understood by reference to the following detailed description, including the drawing and the examples. It should be understood, however, that the detailed description, the drawing and the specific examples, while indicating specific embodiments of the invention, are given by way of illustration only, because various changes and modifications within the spirit and scope of the invention will become apparent to those skilled in the art from this detailed description.

### BRIEF DESCRIPTION OF THE DRAWING

FIG. 1: A: RPMI8226 cells were treated with MG-132 (400 nM) or Bortezomib (Velcade®, VEL) (4 nM) for 24-48 hours and stained with propidium iodide (PI). The cell cycle was analyzed by flow cytometry. The graph shows the Sub-G₀-G₁ fraction. B: Counting of control RPMI8226 cells and cells surviving proteasome inhibition with 400 nM MG-132 for 24 hours using trypan blue exclusion. Note that the cells surviving the treatment do not resume growth for up to a week. C: RPMI8226 cells surviving proteasome inhibition were stained with PI at the indicated time points after drug washout and the cell cycle profile was measured by flow cytometry. Cell cycle profile (upper panel) and quantification (lower panel), *p<0.0015 for all time points, unpaired t-test. D: RPMI8226 cells surviving VEL (4 nM) treatment were stained with PI at 72 hours after drug washout. The graph (upper panel) shows the G₀-G₁ fraction, *p=0.008. Western blots for phosphorylated (p-Rb) and total (Rb) Rb protein in cells surviving proteasome inhibition 72 hours after drug washout (lower panel). β-tubulin was used as a loading control.

FIG. 2: A: RPMI8226 control or pretreated (MG-132 400 nM or Velcade® (VEL) 4 nM) cells were labeled with CFSE for 15 minutes, washed and analyzed by flow cytometry immediately (light gray) or 72 hours later (dark gray). Graphs show a representative result for cells surviving MG-132. B: Quantification of CFSE labeling. Gates for CFSE-positive were set between 10³ and 10⁴ FLH2 (FITC) channel intensity. *p=0.012 for MG and p=0.0002 for VEL, unpaired t-test.

FIG. 3: A-B: Western Blots showing phosphorylation of eIF2α at Ser-51 (A) acutely at 4-8 hours post-exposure and (B) acutely at 24 hours, left two lanes. EIF2α phosphorylation does not persist after drug washout at 72 hours, right two lanes. C: RT-PCR showing increased XBP-1 mRNA splicing and CHOP mRNA expression in the acute phase. Note that splicing of XBP-1 and induction of CHOP are not maintained after drug washout at 72 hours. D-E: Western Blots showing increased expression of Grp78/BiP in the acute phase of proteasome inhibition with 400 nM MG-132 (D) or 4 nM Velcade® (VEL) (E). As opposed to p-eIF2α, CHOP and XBP-1, this induction persists after drug washout for both proteasome inhibitors.

FIG. 4: A: Viability curve in RPMI8226 cells in response to Salubrinal treatment. Note that 5 μM Salubrinal caused no significant induction of cell death (left). B: Western blots for p-eIF2α, eIF2α, p-PERK, PERK, p-GCN2, GCN2, p-PKR and PKR in RPMI8226 cells 8 hours after treatment with Salubrinal, Velcade® (VEL) and the combination of both. Note that phosphorylation of eIF2α via Salubrinal treatment and VEL was further enhanced by the combination of both drugs at 8 hours in RPMI8226 cells while the effect on the upstream kinases PERK, GCN2 and PKR was only marginal. C: Western blots showing that, at 24 hours, both MG 132 and VEL lead to eIF2α proteolytic processing (*), which was shown to be caspase-3 dependent. The effect was enhanced by the addition of Salubrinal. D: Western Blots in U266B1 cells showing increased eIF2α phosphorylation with Salubrinal, VEL and the combination of both (12 hours). Note that, at 12 hours, the addition of Salubrinal to VEL resulted in enhanced eIF2α proteolytic processing (*), while the effect plateaus at 24 hours where similar eIF2α processing with VEL and the VEL/Salubrinal combination was observed. E: RT-PCR showing increased CHOP mRNA levels with 4 nM VEL for 8 hours that were further enhanced by combination of VEL with SAL. GAPDH was used as a loading control.

FIG. 5: A: Percent dead cells in RPMI8226 (left panel) and U266B1 (right panel) after 24 hours of the indicated treatments with Salubrinal (SAL) and/or proteasome inhibitors (MG or Velcade®, VEL) (left). B: RPMI8226 cells transfected with a phosphomimetic S51D eIF2α mutant showed enhanced VEL sensitivity as compared to the empty vector control. The inset shows increased total protein levels of eIF2α in the cells expressing the construct. C: Viability of RPMI8226 cells after 24 hours of the indicated treatments. Cells were treated with either DMSO or VEL 4 nM for 24 hours, then washed and treated with DMSO or Salubrinal (SAL). The right two columns are combination treatments with proteasome inhibitors and Salubrinal for 24 hours as controls. D: The enhanced sensitivity to Salubrinal observed 3 days post-treatment could still be observed at 5 days after proteasome inhibitor washout, indicating that the sensitivity to Salubrinal post-proteasome inhibition was long lasting.

FIG. 6: Quantification of Western blots from FIG. 3: A: FIG. 3A. B: FIG. 3B. C: FIG. 3C. D: FIG. 3D. E: FIG. 3E.

FIG. 7: Quantification of Western blots from FIGS. 4-5: A-D: FIG. 4B, activation of eIF2α (A), PERK (B), GCN2 (C), PKR (D). E: FIG. 4E, activation CHOP mRNA. F: FIG. 5B, overexpression of eIF2αS51D.

FIG. 8: Schematic representation of the effect of proteasome inhibitors and proteins that interfere with GADD34-eIF2α interaction.

### DETAILED DESCRIPTION

Unless otherwise defined, all technical and scientific terms used herein have the same meaning as commonly understood by one of ordinary skill in the art to which this invention belongs. The following references provide one of skill with a general definition of many of the terms used in this invention: Singleton, et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger, et al. (eds.), Springer Verlag (1991); and Hale and Marham, THE HARPER COLLINS DICTIONARY OF BIOLOGY (1991).

It is noted here that, as used in this specification and the appended claims, the singular forms “a,” “an,” and “the” include plural reference unless the context clearly dictates otherwise.

As used herein, the following terms have the meanings ascribed to them unless specified otherwise.

“Treating” means improving, curing, or reducing the severity of a condition or disorder, consistent with its ordinary and accustomed meaning. “Ameliorating” means reducing the degree or severity of, consistent with its ordinary and accustomed meaning. “Preventing” means inhibiting the occurrence, or to keep from happening or existing, consistent with its ordinary and accustomed meaning.

“Administering” is given its ordinary and accustomed meaning of delivery by any suitable means recognized in the art. Exemplary forms of administering include oral delivery, anal delivery, direct puncture or injection, topical application, and spray (e.g., nebulizing spray), gel or fluid application to an eye, ear, nose, mouth, anus or urethral opening.

An “effective combination” is that amount or dose of a combination of compounds that provides a beneficial effect on the organism receiving the combinations and may vary depending upon the purpose of administering the combination, the size and condition of the organism receiving the dose, and other variables recognized in the art as relevant to a determination of an effective combination. The process of determining an effective combination involves routine optimization procedures that are within the skill in the art.

An “animal” is given its conventional meaning of a non-plant, non-protist living being. In various aspects, the animal is a mammal. In more particular aspects, the mammal is a human. In other aspects, the mammal is a human pet or companion animal, a domesticated farm animal, or a zoo animal.

In the context of the present disclosure, a “need” is an organismal, organ, tissue, or cellular state that could benefit from administration of an effective combination to an organism characterized by that state. For example, a human at risk of developing cancer, or presenting a symptom thereof, is an organism in need of an effective combination of a product, such as a pharmaceutical composition, according to the present invention.

“Inhibiting” is given its ordinary and accustomed meaning of inhibiting, reducing or preventing. For example, inhibiting morphological change means that morphological change is made more difficult or prevented entirely.

A “proteasome inhibitor” is a drug or compound that blocks the action of proteasomes, cellular complexes that break down proteins. In more particular aspects, a proteasome inhibitor includes, but is not limited to, bortezomib (Velcade®, Millenium Pharmaceuticals, Inc.), MG 132, disulfiram, epigallocatechin-3-gallate, and salinosporamide A. One of skill in the art understands other proteasome inhibitors that are contemplated for use herein.

“Salubrinal” is a cell-permeable, selective inhibitor of cellular phosphatase complexes that dephosphorylate eukaryotic translation Initiation Factor 2 subunit α (eIF2α). Salubrinal protects cells from endoplasmic reticulum stress-induced apoptosis. Salubrinal is available from Alexis Biochemicals or Tocris Bioscience (Cat No. 2347), or other source as known to one of skill in the art.

Eukaryotic Initiation Factor 2 or eukaryotic translation Initiation Factor 2 (eIF2) is a GTP-binding protein responsible for bringing the initiator tRNA to the P-site of the pre-initiation complex. eIF2 has specificity for the methionine-charged initiator tRNA, which is distinct from other methionine-charged tRNAs specific for elongation of the polypeptide chain. Once it has placed the initiator tRNA on the AUG start codon in the P-site, it hydrolyzes GTP into GDP, and dissociates. This hydrolysis, also signals for the dissociation of eIF3, eIF1, and eIF1A, and allows the large subunit to bind. This signals the beginning of elongation. eIF2 has three subunits, eIF2-α, β, and γ. “Eukaryotic Initiation Factor 2-α” or “eIF2-α” is of particular importance for cells that may need to turn off protein synthesis globally. When phosphorylated, eIF2-α sequesters eIF2B (not to be confused with beta), a GEF. Without this GEF, GDP cannot be exchanged for GTP, and translation is repressed. eIF2α-induced translation repression occurs in reticulocytes when starved for iron. In addition, protein kinase R (PKR) phosphorylates eIF2α when dsRNA is detected in many multicellular organisms, leading to cell death.

“Growth Arrest and DNA Damage-Inducible Protein 34” or “GADD34” is a stress-induced protein implicated in the control of protein synthesis and apoptosis. GADD34 is a major target of the oncogene c-myc.

“Glucose-regulated protein 78” or “Grp78” or “GRP78” is a key regulator of the unfolded protein response (UPR). As a Ca²⁺-binding molecular chaperone in the endoplasmic reticulum (ER), Grp78 maintains ER homeostasis, suppresses stress-induced apoptosis, and controls UPR signaling. All proteins or compounds that inhibit Grp78 or the biological function of Grp78 are used in the context of the disclosure.

In the context of the present disclosure, “cancer” is a class of diseases in which a group of cells display uncontrolled growth, invasion, and sometimes metastasis via lymph or blood. These three malignant properties of cancers differentiate them from benign tumors, which are self-limited, and do not invade or metastasize. In the context of the present disclosure, “cancer” is given its ordinary and accustomed meaning and includes all diseases that are classified as a cancer. The terms “cancer” or “cancerous condition” are used interchangeably herein, and all types of cancer are included in the methods of treatment herein.

In one aspect, a hematological malignancy is one of the types of cancer that is treated or prevented in the methods of the invention. A “hematological malignancy” is a type of cancer that affects blood, bone marrow, and lymph nodes. As the three are intimately connected through the immune system, a disease affecting one of the three will often affect the others as well. Hematological malignancies may derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid line, while acute and chronic myelogenous leukemia, myelodysplastic syndromes and myeloproliferative diseases are myeloid in origin.

“Multiple myeloma,” also known as “MM,” “myeloma,” “plasma cell myeloma,” or as “Kahler's disease” (after Otto Kahler) is part of the broad group of diseases called hematological malignancies. “Multiple myeloma” is a cancer of the white blood cells known as plasma cells. A type of B cell, a plasma cell is a crucial part of the immune system responsible for the production of antibodies in humans and other vertebrates. Plasma cells are produced in the bone marrow and populate, and are transported through, the lymphatic system. Myeloma is part of the broad group of diseases called hematological malignancies.

The phrase “therapeutically effective combination” or “prophylactically effective combination” refers to the amount or dose of the combination of proteasome inhibitor and protein sufficient to interfere with the functional interaction of GADD34 and eukaryotic Initiation Factor 2α or to inhibit Grp 78. This “therapeutically effective combination” or “prophylactically effective combination” results in any amelioration, treatment, prevention or alteration of any biological symptom generally associated with cancer including, without limitation, multiple myeloma.

“Interaction” is given its ordinary and accustomed meaning of interplay, as in the interplay between or among two or more biological products, such as molecules, cells, and the like. Molecules include, but are not limited to, nucleic acids and peptides or polypeptides.

“Pharmaceutical composition” or “composition” means a formulation of compounds suitable for prophylactic or therapeutic administration, to a living mammal, such as an animal or human patient. The composition may comprise one or more pharmaceutically acceptable carriers. The phrase “pharmaceutically or pharmacologically acceptable” refers to molecular entities and compositions that do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like.

The compositions are administered, for example and without limitation, orally, topically, transdermally, parenterally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and or surgical implantation at a particular site is contemplated as well. In certain aspects, compositions are essentially free of pyrogens, as well as other impurities that could be harmful to the recipient.

Formulation of the compositions vary according to the route of administration selected (e.g., solution or emulsion). An appropriate composition comprising the compound, e.g., inhibitor and/or protein, and the like, to be administered can be prepared in a physiologically acceptable vehicle or carrier. For solutions or emulsions, suitable carriers include, for example and without limitation, aqueous or alcoholic/aqueous solutions, emulsions or suspensions, including saline and buffered media. Parenteral vehicles include, for example and without limitation, sodium chloride solution, Ringer's dextrose, dextrose and sodium chloride, lactated Ringer's or fixed oils. Intravenous vehicles include, for example and without limitation, various additives, preservatives, or fluid, nutrient or electrolyte replenishers.

The compositions are, in various embodiments, lyophilized for storage and reconstituted in a suitable carrier prior to use. Any suitable lyophilization or reconstitution techniques is employed. It will be appreciated by those skilled in the art that lyophilization and reconstitution can lead to varying degrees of antibody activity loss and that use levels may have to be adjusted to compensate.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above.

The pharmaceutical compositions in various aspects are in the form of a sterile injectable aqueous fluid, oleaginous suspension, dispersion or sterile powder for the extemporaneous preparation of a sterile injectable solution or dispersion. The suspension is formulated according to the art using those suitable dispersing or wetting agents and suspending agents which have been mentioned above. The sterile injectable preparation includes sterile injectable solutions or suspensions in a non-toxic parenterally acceptable diluent or solvent, for example as a solution in 1,3-butanediol. The carrier is in certain aspects a solvent or dispersion medium containing, for example, water, ethanol, polyol (for example, glycerol, propylene glycol, liquid polyethylene glycol, and the like), suitable mixtures thereof, vegetable oils, Ringer's solution and isotonic sodium chloride solution. In addition, sterile, fixed oils are conventionally employed as a solvent or suspending medium. For this purpose, any bland fixed oil is employed including synthetic mono- or diglycerides. In addition, fatty acids such as oleic acid find use in the preparation of injectables.

In all cases, the form must be sterile and must be fluid to the extent that easy syringability exists if administration by injection is employed. The proper fluidity is maintained, for example, by the use of a coating, such as lecithin or other coating well known in the art, by the maintenance of the required particle size in the case of dispersion and by the use of surfactants. It must be stable under the conditions of manufacture and storage and must be preserved against the contaminating action of microorganisms, such as bacteria and fungi. The prevention of the action of microorganisms is brought about by various antibacterial and antifungal agents, for example, parabens, chlorobutanol, phenol, sorbic acid, thimerosal, and the like. In many cases, it will be desirable to include isotonic agents, for example, sugars or sodium chloride. Prolonged absorption of the injectable compositions is brought about by the use in the compositions of agents delaying absorption, for example, aluminum monostearate and gelatin.

Proteasome inhibitors effectively eradicate multiple myeloma (MM) cells, partly by activating endoplasmic reticulum (ER) stress apoptotic signaling. However, MM recurrences in treated patients are invariable. ER stress signaling can also induce growth arrest and survival in cancer cells. Thus, it was expected that proteasome inhibitor therapy would induce quiescence and survival of residual MM cells, contributing to disease recurrence. In MM cells, proteasome inhibition resulted in a surviving cell fraction that entered a prolonged quiescent state (G₀-G₁ arrest). Mechanism analysis revealed that proteasome inhibitor-surviving quiescent cells attenuated eIF2α phosphorylation and induction of the ER stress pro-apoptotic gene, GADD153. In contrast, the pro-survival ER-chaperone BiP/Grp78 was persistently induced. The proteasome inhibitor-surviving quiescent fraction could be eradicated by a simultaneous or sequential combination therapy with an inhibitor of the GADD34-PP1C phosphatase complex (e.g., Salubrinal) and, as a consequence, eIF2α dephosphorylation. This effect was mimicked by expression of a phospho-mimetic eIF2α-S51D mutant. Thus, it is expected that growth arrest and survival may be a response to proteasome inhibitor therapy. The data disclosed herein indicate that proteasome inhibitors induce growth arrest in therapy-surviving MM cells and that attenuation of eIF2α phosphorylation contributes to this survival. Importantly, this survival mechanism can be blocked by inhibiting eIF2α dephosphorylation. Thus, strategies that maintain eIF2α in a hyper-phosphorylated state provide a therapeutic approach to maximize proteasome inhibitor-induced apoptosis and reduce residual disease and recurrences in this and related types of cancer.

Any link between proteasome inhibition, activation of ER stress and growth arrest in MM cells was examined. It was expected that proteasome inhibition with MG-132 or Bortezomib in clinically relevant concentrations may force cells into a growth arrest/survival program due to an ER stress adaptation response. This could be a potential contributor to therapy resistance and disease recurrence. It was further expected that maximizing ER stress signaling could enhance sensitivity to proteasome inhibitors by tipping the balance from signaling for growth arrest/survival to apoptosis. As disclosed in the Examples, a fraction of MM cells surviving a single treatment with Bortezomib downregulate eIF2α phosphorylation and enter a prolonged G₀-G₁ arrest. More importantly, the quiescent surviving fraction of MM cells with different genetic abnormalities could be almost completely eradicated by enhancing eIF2α phosphorylation with Salubrinal (an inhibitor of GADD34-PPc assembly or GADD34-eIF2α interaction) or by expression of a phospho-mimetic eIF2αS51D protein. The data disclosed herein establish that enhancement of ER stress signaling can be exploited as a strategy to maximize efficiency of proteasome inhibitor therapy.

Exploring mechanisms that might induce quiescence in cancer cells led to the study of whether therapies known to induce the Unfolded Protein Response (i.e., UPR) might be the cause of a protective response in cancer cells. It had previously been shown that head and neck cancer cells (HNSCC) entered a state of cellular dormancy that was linked to the activation of ER stress pathways that promoted both growth arrest and survival. These are the same pathways that can be activated by proteasome inhibitors, indicating that proteasome inhibition with MG-132 and the clinically used drug Bortezomib (Velcade®) should induce quiescence and persistence of cells surviving the therapy.

Several findings are worth noting. While a portion of MM cells treated with proteasome inhibitors died, a significant fraction (30-50% of the cells) could adapt to the stress and survive by entering a protracted state of quiescence. The survival of about 30-50% of the cells indicated that the G₀-G₁ arrest is more likely due to treatment adaptation than to selection of a cell population genetically predisposed to undergo quiescence. This response is plausible as one function of ER stress signaling is to allow cells to pause proliferation while unfolding of proteins in the ER is being corrected. This ultimately leads to cell survival and, in the context of MM and related cancers, may allow for recurrence.

Survival of the residual quiescent cells hinges on the downregulation of eIF2α phosphorylation, which allows these cells to silence pro-apoptotic signals downstream of eIF2α signaling. This was primarily evidenced by the loss of CHOP mRNA expression. The silencing of CHOP provides an important apoptosis escape mechanism, as this transcription factor was shown to repress Bcl-2 expression and, at the same time, induce expression of BimEL, a powerful pro-apoptotic protein. A solution to this problem was found by combining Bortezomib treatment with Salubrinal, an inhibitor of GADD34-PP1c complex assembly. Maintaining eIF2α phosphorylation through drugs like Salubrinal can potentiate killing of MM cells and they may serve as an adjuvant therapy to proteasome inhibitors when administered simultaneously. Second, Salubrinal can virtually eliminate the fraction of quiescent MM cells surviving proteasome inhibition. It is therefore expected that Salubrinal and similar compounds affecting phosphorylation of eIF2α will serve as elements in strategies to maximize proteasome inhibitor efficiency and to decrease MM cell resistance to this substance class. The ability of Salubrinal to potentiate killing of MM cells by enhancing eIF2α phosphorylation is in accordance with previous models that set up a sigmoidal relationship between stress intensity and eIF2α activation, leading to survival at very low levels and apoptosis at very high levels. Thus, the enhancement of eIF2α phosphorylation during MG-132 or Bortezomib treatment prevents MM cells from modulating this pathway to adapt to, and survive, ER stress.

This response was observed in two MM cell lines with different genetic alterations (c-MYC insertion on t(16;22)(q32;q11):der16 in RPMI8226 and t(11;14) in U266B1), indicating that it may not be linked to a given genotype. Also, it was discovered that, while diffuse B-cell lymphoma U937 cells were highly sensitive to 4 nM Bortezomib and K-562 chronic myeloid leukemia cells were intermediately sensitive, neither of these cell lines was susceptible to an enhancement of Bortezomib-induced killing by Salubrinal. Further, neither Bortezomib alone nor its combination with Salubrinal was effective in inducing killing of HEp3 HNSCC or Raji Burkitt's lymphoma cells. This indicated that the combination therapy using a proteasome inhibitor and Salubrinal may be primarily suited for the treatment of multiple myeloma and related cancers.

It is expected that the induction of XBP-1 during the acute phase of Bortezomib treatment is associated with a survival/adaptation response. It is possible that while XBP-1 may function to protect cells during the acute phase, its ability to lead to the prolonged induction of chaperones such as BiP/Grp78, may prevent apoptosis in the cells surviving the treatment. This response may be related to the inherent capacity of the UPR to protect from subsequent insults to the ER. Thus, prolonged survival of the quiescent fraction may be linked to the abundant induction of BiP/Grp78. Previous studies suggested a pro-survival function for this chaperone in cancer. Thus, it is expected that, in addition to the inhibition of GADD34-PP1c and enhancement of p-eIF2α signaling, blockade of Grp78 expression and/or function may be an additional strategy to prevent adaptation and survival of MM and other cancer cells to proteasome inhibitor treatment. Strategies similar to those applied to identify HSP90 inhibitors are expected to be successful in identifying small molecule inhibitors specific for Grp78.

Several upstream kinases, such as PERK, PKR and GCN2, can phosphorylate eIF2α. In particular, during the acute phase there was a slight enhancement of p-PERK and p-PKR levels, but not of p-GCN2. However, it is still not clear whether these kinases contribute to growth arrest. It is also unclear whether the downregulation of eIF2α phosphorylation is solely due to GADD34-PP1c expression/activity or to reduced activity of the upstream kinases. What is evident from the results disclosed herein is that, in both acute and post-Bortezomib treatments with Salubrinal, strong phosphorylation of eIF2α is associated with apoptosis. Studies in HNSCC cells showed that while PERK and eIF2α signaling can contribute to survival, persistent eIF2α signaling is also linked to the quiescent state. In the case of MM cells, phosphorylation of eIF2α does not persist post-treatment during the quiescence phase. This indicated that the growth arrest was linked to other mechanisms or that persistent eIF2α phosphorylation was not needed for this effect.

While Rb phosphorylation in sites that are indicative of G₁-exit was reduced after proteasome inhibition, no significant changes in CDK or cyclin protein levels were detected. Only p21 induction was observed in the acute phase but not in the surviving fraction of arrested cells. It was expected that this acute induction is sufficient to induce the long-term MM cell arrest and that this or other mechanisms (i.e., p15, p16 induction) maintain Cyclin/CDK complexes in inactive states for prolonged periods.

Thus, the induction of MM tumor cell quiescence and survival is an undesirable side effect of proteasome inhibition. Data disclosed herein establish that by blocking eIF2α dephosphorylation, proteasome inhibitor efficiency can be maximized during acute treatment and that residual cells can be eliminated by non-toxic doses of either a protein that interferes with GADD34-PP1c complex assembly (e.g., Salubrinal) or a peptide that inhibits Grp78 function as a monotherapy for MM, or like cancer, minimal residual disease following proteasome inhibition.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Example 1

### Materials and Methods

Reagents, antibodies, RT-PCR, cell lines and tissue culture, plasmids. Proteasome inhibitor (MG-132, Velcade®; each commercially available) treatments were performed using 400 nM MG-132 or 4 nM Bortezomib (Velcade®) for 24 hours. The drug was then removed by serial washes with PBS and the remaining viable cells were replated. Salubrinal treatments were performed using the drug at 5 or 10 μg/ml, as indicated, for 24 hours. RPMI8226 cells contain a c-MYC insertion on t(16;22)(q32;q11):der16; U266B1 cells (t(11;14)) were from ATCC. Cells were cultured according to ATCC recommendations. Antibodies (identified by the antigen specifically recognized): p-Rb, Rb, p-eIF2α, eIF2α, GCN2, p-GCN2, p-PKR and PKR from Cell Signaling (Beverly, Mass.), p-PERK and PERK from Santa Cruz Biotechnology (Santa Cruz, Calif.), BiP from BD (San Jose, Calif.), GAPDH from Calbiochem (Beverly, Mass.), and β-tubulin from Abcam (Cambridge, Mass.). Total RNA was extracted using Trizol (Invitrogen, Carlsbad, Calif.). Primer sequences were published previously (Sequeira, et al., PLoS ONE 2(7):e615 2007, incorporated herein by reference in its entirety). The pCLBabe-eIF2αS51D plasmid was provided by Dr. David Schubert (Salk Institute, La Jolla, Calif.) and can be constructed using known techniques. Transfection of RPMI8226 cells was performed using Amaxa technology according to the manufacturer's instructions (Gaithersburg, Md.).

Cell cycle analysis, label retention assay. Cell cycle analysis was performed using propidium iodide (PI)/RNAse staining buffer from BD according to the manufacturer's instruction. For label retention and viability quantification, cells were treated with proteasome inhibitors or DMSO for 24 hours, then stained with 50 nM CFDA/SE (Molecular Probes, Eugene, Oreg.) for 15 minutes and replated. Prior to analysis, cells were stained using Hoechst-33342 (10 μg/ml) for 15 minutes at 4° C. to determine cell viability. Cells were then fixed using 3% paraformaldehyde for 20 minutes and analyzed by flow cytometry (LSR-II from BD).

Viability assay, Immunoblotting. Cell viability was assessed using trypan blue. Immunoblotting was performed using convention techniques, as described previously Aguirre-Ghiso, et al., Cancer Res. 63(7):1684-1695 2003, incorporated herein by reference in its entirety.

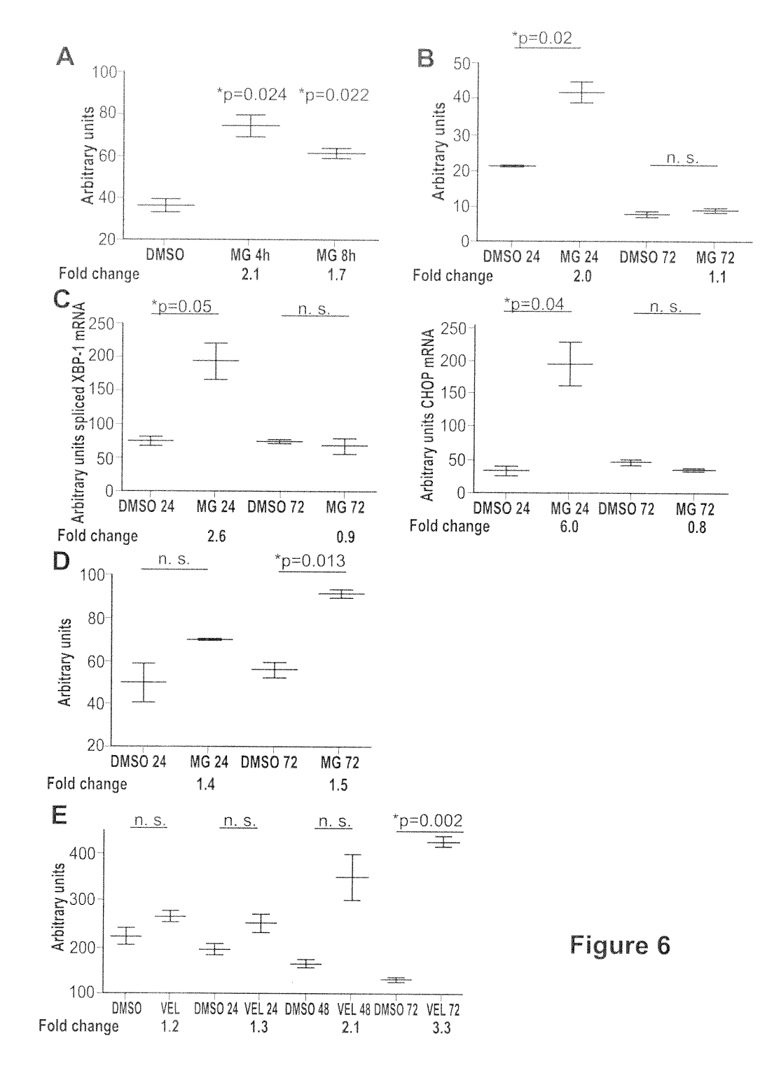

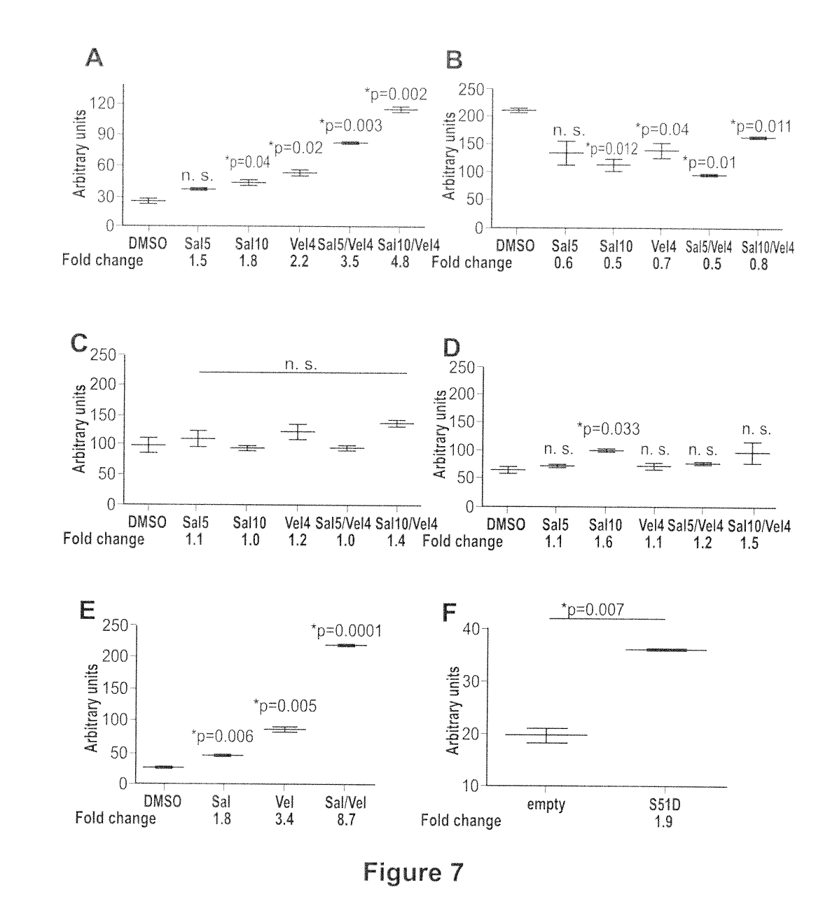

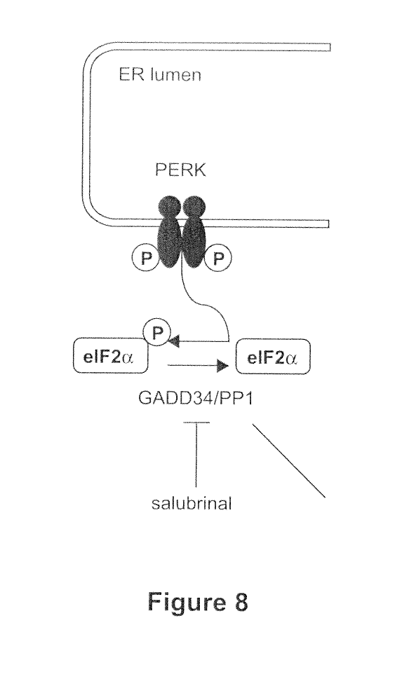

Image quantification and statistical analysis. Image quantification was performed using ImageJ from NIH. Quantification of all Western blots and RT-PCR experiments showing changes in expression levels are given in FIGS. 6 and 7. Statistical analyses were performed using Prism 4.0 from GraphPad Software (La Jolla, Calif.) and the Student's t-test.

### Example 2

### Bortezomib Induces G₀-G₁ Arrest in a Fraction of Surviving Cells

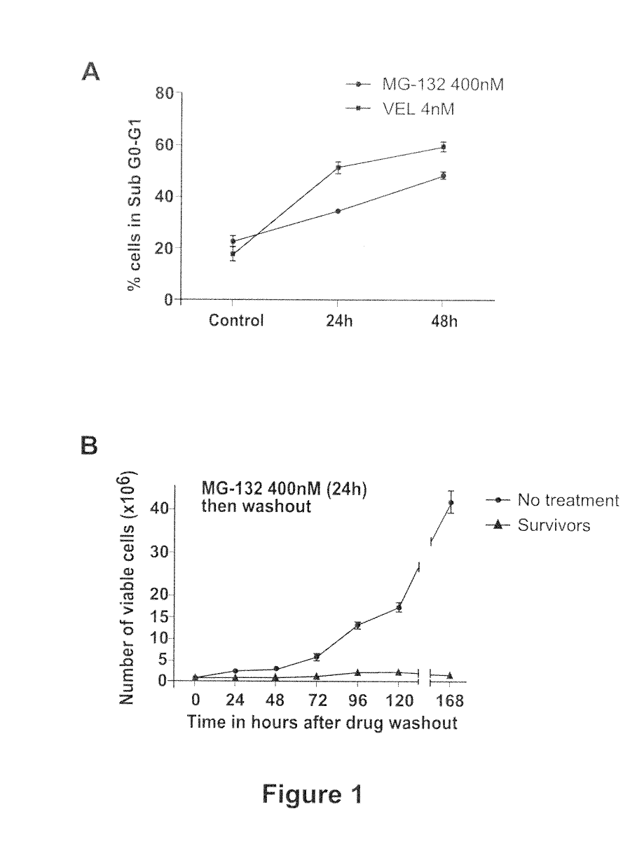

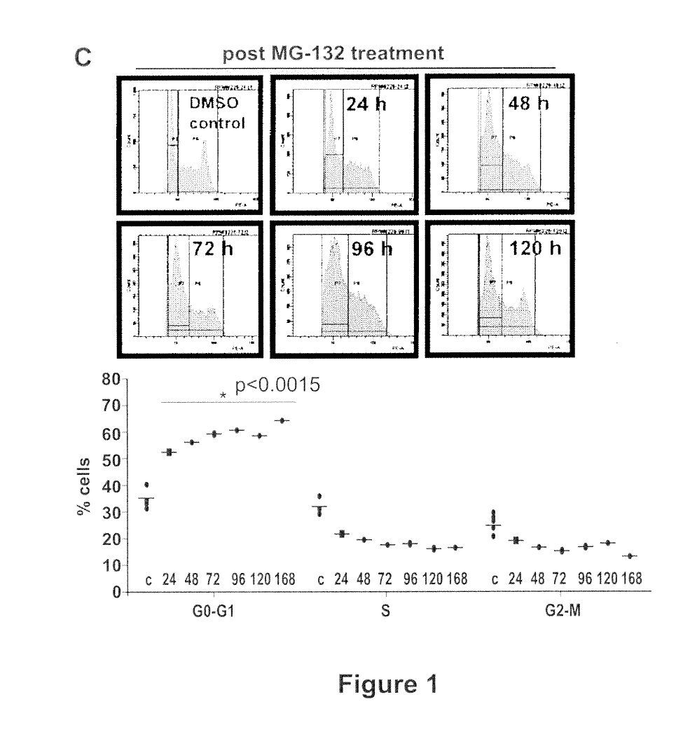

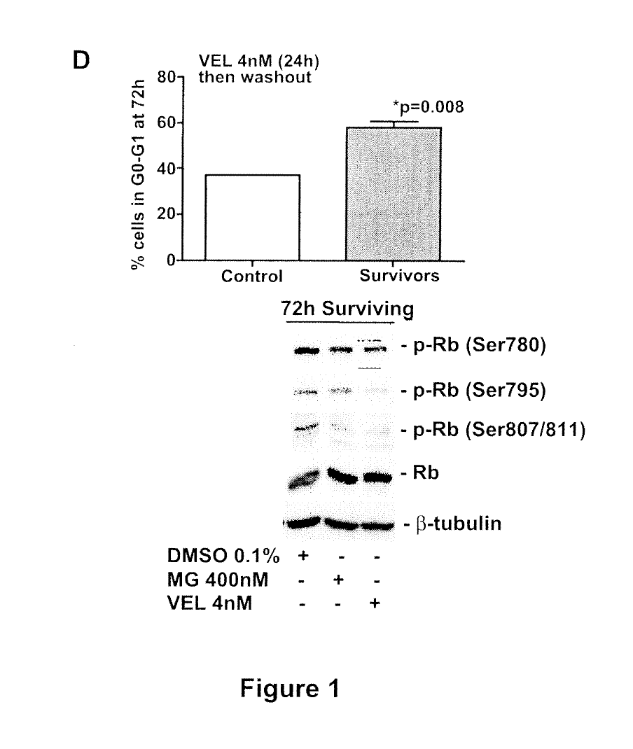

Treatment of RPMI8226 MM cells with MG-132 (400 nM) and Bortezomib (4 nM) strongly induced cell death (sub-G₀ population) in a time-dependent fashion (FIG. 1A). This may be due to an initial (4-8 hours) G2-M arrest as determined by FACS and in accordance with data in other systems. The fate of MM cells surviving proteasome inhibitor therapy was explored. RPMI8226 cells treated with 400 nM MG-132 for 24 hours were washed to remove the drug. The absolute number of viable cells that were then replated remained constant for 7 days. In contrast, vehicle-treated cells initiated exponential growth after 48 hours (FIG. 1B). The lack of expansion in the surviving cells was not due to a balance between proliferating and dying cells, but to a growth arrest as shown by propidium iodide (PI) staining followed by flow cytometry (FIG. 1C). Cell cycle analysis revealed that the number of cells in G₀-G₁ increased significantly from about 35% in the control population to about 55-65% in the cells surviving proteasome inhibition (FIG. 1C). Similar results were obtained using 4 nM Bortezomib, a dose that is pharmacodynamically achievable in patients (FIG. 1D, upper panel). The arrest was due to a failure to transit the G₁-S boundary because 72 hours after washout of proteasome inhibitors, there was a strong reduction in phosphorylated Rb protein levels. This happened at Ser residues that were phosphorylated to promote G₁-S transition (FIG. 1D, lower panel). These results showed that two proteasome inhibitors were able to induce G₀-G₁ arrest in RPMI8226 MM cells after a single treatment.

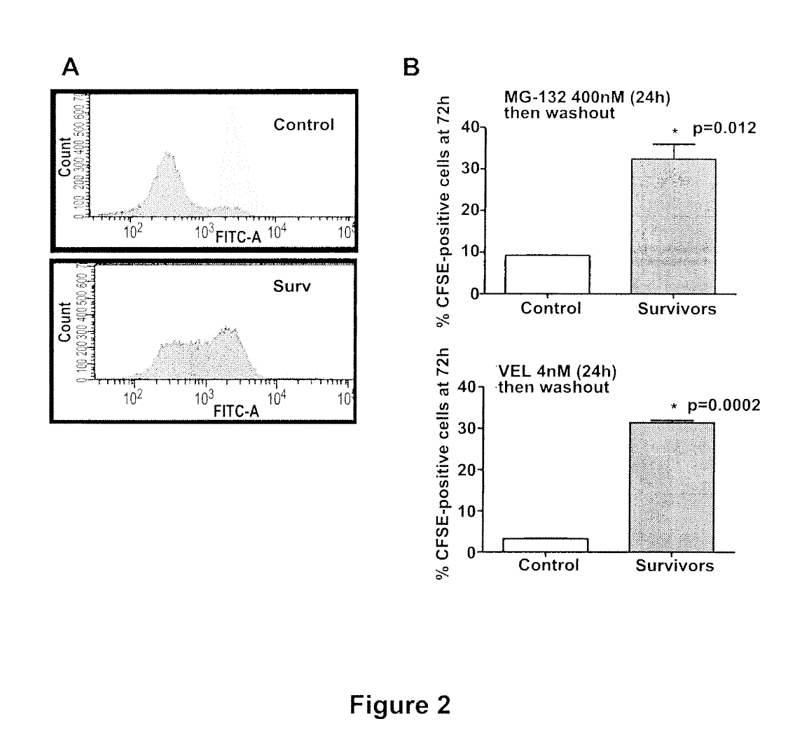

To corroborate the cell cycle analysis, double labeling with CFSE (carboxyfluorescein succinimidyl ester) and Hoechst-33342 was performed to measure viable CFSE-retaining cells and to gate out sub-G0 cells. CFSE, due to its irreversible esterification to cellular proteins and its equal distribution within daughter cells, can be used to monitor cell division. A highly significant approximately 4-7-fold increase in the percentage of CFSE-positive cells was found at 72 hours in cells surviving both MG-132 and Bortezomib (FIG. 2A-B), indicating that, at 72 hours after the washout of proteasome inhibitors, the surviving cells divided to a lesser extent. These results indicated that while proteasome inhibition can cause acute apoptosis, the surviving MM cells rapidly enter a G₀-G₁ arrest. Given that pharmacologic treatments do not reach maximal lethal doses in all targeted cells, the induction of a growth-arrested surviving fraction adapting to the treatment may be a side effect of proteasome inhibition.

### Example 3

Bortezomib-Surviving Cells Attenuate EIF2α Phosphorylation and Uncouple the Induction of Pro-Apoptotic from Pro-Survival ER Stress Genes

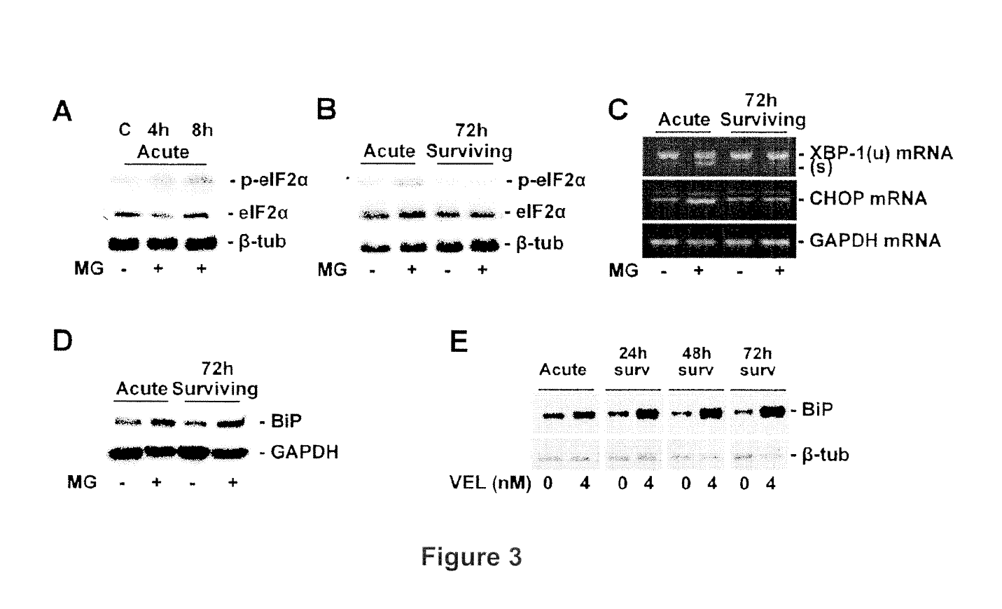

Next, the cells surviving proteasome inhibitors were examined to determine if any undergo any specific changes in ER-stress signaling. Proteasome inhibitors are known to induce an unfolded protein response in MM cells by inhibition of the ER associated degradation pathway. Further, the unfolded protein response (UPR) mediates cell survival and drug resistance in several models, including MM. Interest focused on phosphorylation of eIF2α, which attenuates translation and induces a selective gene expression program as an adaptive response to ER-stress. Also monitored was the expression of the pro-survival chaperone BiP/Grp78, the transcription factor XBP-1 and the pro-apoptotic transcription factor CHOP. MG-132 treatment caused increased eIF2α-phosphorylation (FIG. 3A-B), XBP-1 splicing (FIG. 3C), BiP (FIG. 3D-E) and CHOP (FIG. 3C) upregulation in the acute phase (8-24 hours). With the exception of BiP, which was strongly induced, the expression of CHOP mRNA, spliced XBP-1, or increased eIF2α phosphorylation did not persist in the surviving cells (FIG. 3A-E). Phosphorylation of p38 was also activated by each of MG-132 and Bortezomib, and p38 remained phosphorylated in the surviving quiescent cells. This indicated that p38 activation can have a pro-survival role in MM cells. BiP is known to be able to induce survival in other cancer cells, indicating that these functions might be preserved during quiescence induced by proteasome inhibitors in MM cells.

Interestingly, both CHOP induction and XBP-1 splicing are only observed during the acute phase and both are then downregulated in the surviving cells. It is possible that downregulation of CHOP eliminates the pro-apoptotic function of this gene, further promoting survival of the residual MM cells.

### Example 4

Pharmacologic or Genetic Enhancement of eIF2α Phosphorylation Potentiates Bortezomib-Induced Cell Death, Minimizing the Surviving Cell Fraction

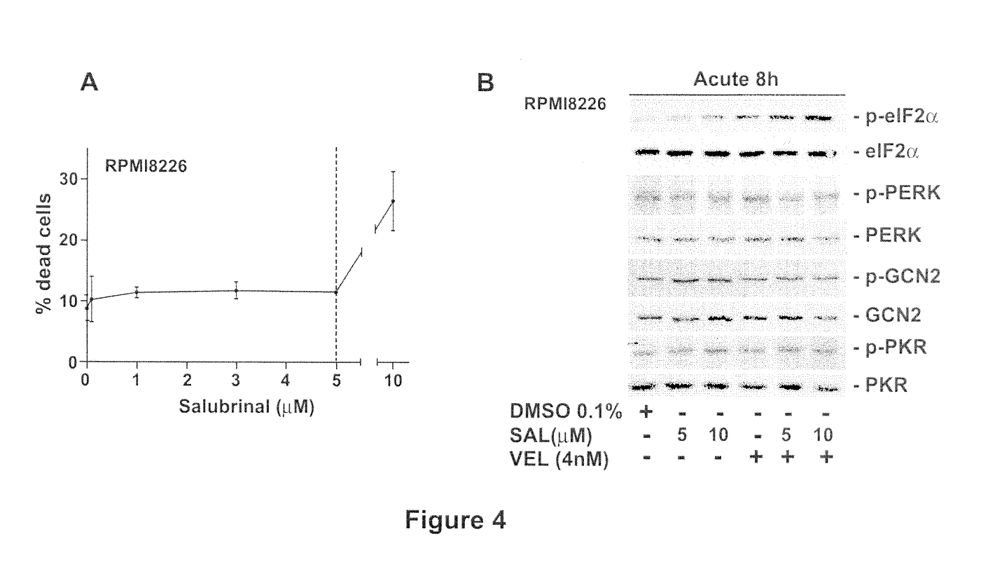

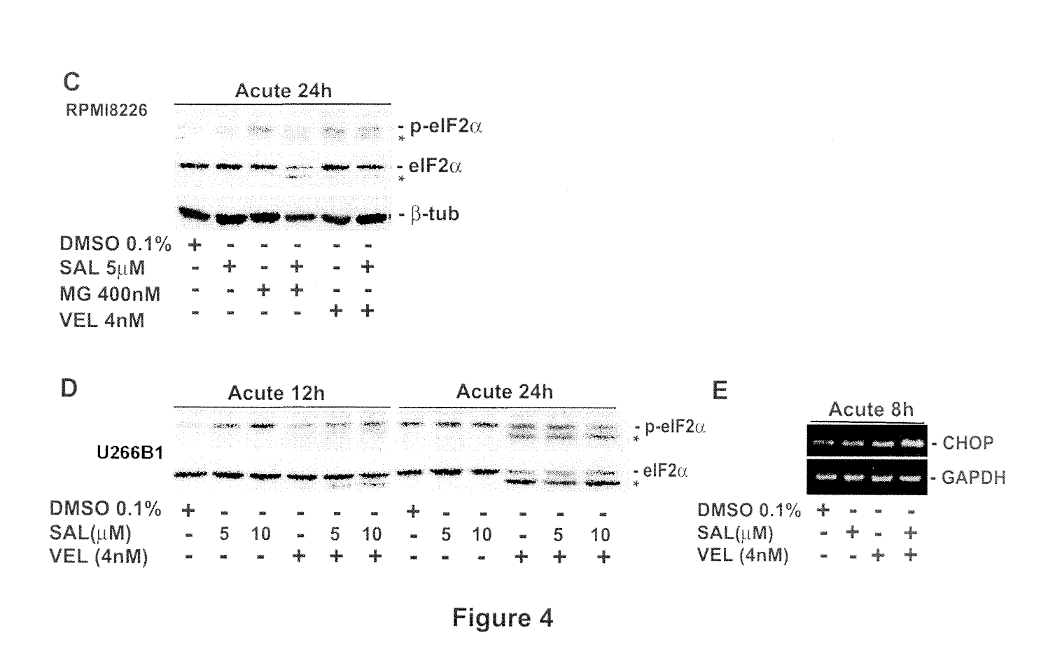

Loss of eIF2α phosphorylation and of its downstream target CHOP in the surviving fraction may be associated with an evasion of apoptosis, as enhanced phosphorylation of this protein can induce cell death. Thus, further testing was carried out to determine whether sustained eIF2α phosphorylation in the surviving cells would limit their survival after Bortezomib treatment. To this end, Salubrinal was used as an inhibitor of GADD34-PP1c complex assembly, and therefore eIF2α-dephosphorylation. Salubrinal was tested to determine if it would enhance Bortezomib cytotoxicity acutely and, importantly, in the fraction of MM cells surviving proteasome inhibitor treatment. At a dose of 5 μM, Salubrinal had no effect on the basal viability or growth of RPMI8226 (FIG. 4A), despite inducing eIF2α phosphorylation (FIG. 4B). Thus, this Salubrinal dose was employed for treating MM cells in combination with Bortezomib or following proteasome inhibitor treatment. This would reveal if maintaining this phosphorylation could potentiate Bortezomib-induced killing in the acute phase and also cause death of the surviving cells. As noted herein, Salubrinal alone resulted in increased phosphorylation of eIF2α at 8 hours. However, proteasome inhibition alone caused even higher p-eIF2α levels than Salubrinal alone (FIG. 4B). Combining Salubrinal and the proteasome inhibitor resulted in the highest levels of p-eIF2α at 8 hours in RPMI8226 MM cells, indicating that this combination can potentiate the activation of this pathway (FIG. 4B). Treatment with Salubrinal, Bortezomib and the combination all decreased the activation of PERK marginally, while no effects could be seen on the activation of GCN2 and PKR was only slightly induced by 10 μM Salubrinal (FIG. 4B, FIG. 7). This indicated that the observed induction of eIF2α phosphorylation by Salubrinal, proteasome inhibitors and the combination of both was mediated to a small extent by the upstream kinase PERK (FIG. 4B). Combinatorial treatment of both RPMI8226 and U266B1 cells for 12 and 24 hours with MG-132 or Bortezomib did not further enhance eIF2α phosphorylation but resulted in pronounced cleavage of eIF2α (not observed at 8 hours). This was reported to depend on caspase-3 activation and is indicative of an irreversible cellular commitment to apoptosis (FIG. 4C-D). Consistent with these findings, acute combination of Salubrinal and Bortezomib also caused a strong induction of the mRNA for the pro-apoptotic gene CHOP (FIG. 4E), which indicated that the enhancement of Bortezomib-induced apoptosis by Salubrinal is due to strong pro-apoptotic mediators regulated by this transcription factor.

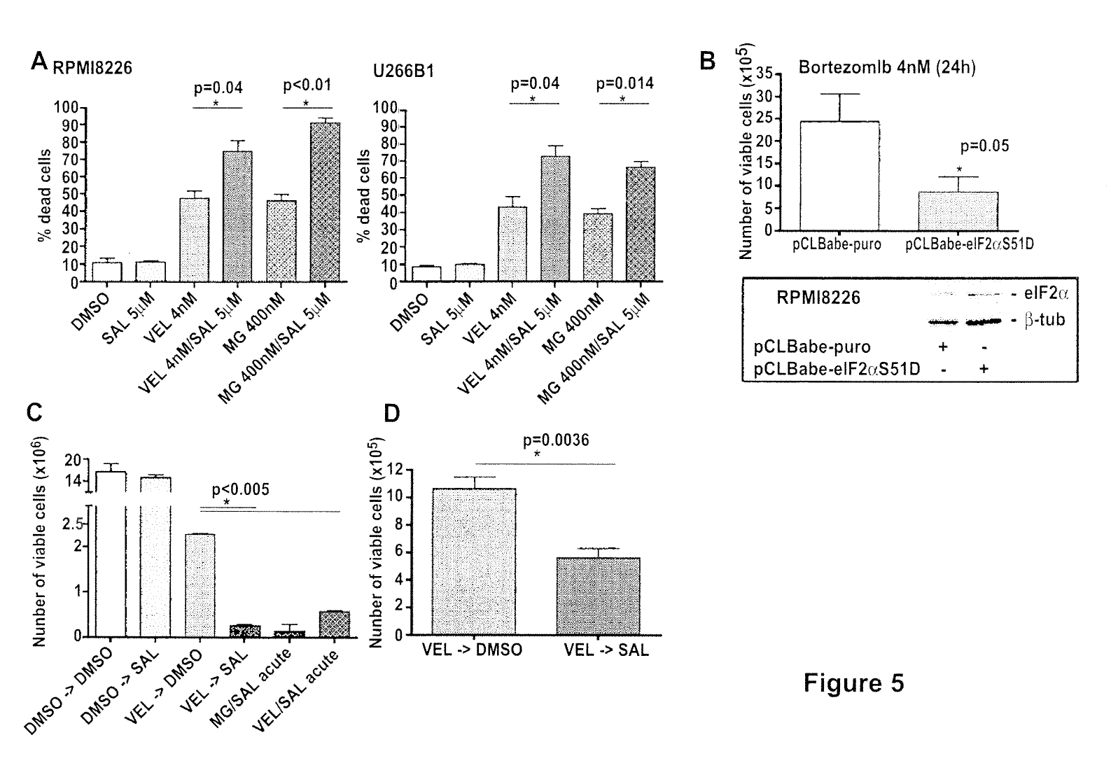

While in both RPMI8226 and U266B1 cells, MG-132 or Bortezomib alone caused roughly 40-50% cell death at 24 hours, Salubrinal could further augment this effect to roughly 70-90% (FIG. 5A), which is in agreement with the activation of apoptotic signals. These studies argue for this compound being highly specific for the GADD34-PP1c complex. The data obtained by transfecting RPMI8226 cells with a phospho-mimetic eIF2αS51D mutant strongly indicated that the effect is specific for GADD34-PP1c activity on eIF2α (FIG. 5B). Transient transfection efficiency in these cells using electroporation is about 50%, and cell death was enhanced by around the same magnitude when combined with proteasome inhibition (FIG. 5B). Thus, it can be deduced that a majority of MM cells expressing the eIF2αS51D mutant were more sensitive to Bortezomib than counterpart wild-type cells.

Salubrinal treatment of the cells that are arrested but still surviving proteasome inhibition was tested for any effect on cell viability. Treatment of this MM cell population with 5 μM Salubrinal, which does not affect the control population, caused an approximately 10-fold reduction in the number of viable cells after 24 hours (FIG. 5C). This reduction was comparable to the effect observed using the combination therapy (FIG. 5C). The effect could still be observed in quiescent cells 5 days after the Bortezomib treatment (FIG. 5D). Thus, these cells, while still growth-arrested, were in a high ER stress condition, evidenced by the high Grp78 levels, and remained highly sensitive to Salubrinal. These results further establish that the reduction in p-eIF2α observed after Bortezomib treatment is indeed a mechanism to evade apoptosis.

The subject matter of the disclosure has been described in terms of particular embodiments found or proposed to comprise specific modes for the practice of the invention. Variations on the subject matter of the disclosure provided herein will be apparent to those of skill in the art upon review of the present disclosure, and such variations are within the scope of the subject matter disclosed.

What is claimed is:

1. A method for treating cancer comprising administering a therapeutically effective combination of a proteasome inhibitor and a protein that interferes with the functional interaction of GADD34 and eukaryotic Initiation Factor 2α.

2. The method according to claim 1 wherein the cancer is multiple myeloma.

3. The method according to claim 1 wherein the proteasome inhibitor is selected from the group consisting of MG 132 and Velcade®.

4. The method according to claim 1 wherein the protein that interferes with the functional interaction of GADD34 and eukaryotic Initiation Factor 2α is salubrinal.

5. The method according to claim 1 wherein the organism is a human.

6. A method for treating cancer comprising administering a therapeutically effective combination of a proteasome inhibitor and a protein that inhibits Grp78 function to an organism in need.

7. The method according to claim 6 wherein the cancer is multiple myeloma.

8. The method according to claim 6 wherein the proteasome inhibitor is selected from the group consisting of MG 132 and Velcade®.

9. The method according to claim 6 wherein the organism is a human.

10. A method for preventing a cancerous condition comprising administering a prophylactically effective combination of a proteasome inhibitor and a protein that interferes with the functional interaction of GADD34 and eukaryotic Initiation Factor 2α to an organism in need.

11. The method according to claim 10 wherein the cancerous condition is multiple myeloma.

12. The method according to claim 10 wherein the proteasome inhibitor is selected from the group consisting of MG 132 and Velcade®.

13. The method according to claim 10 wherein the protein that interferes with the functional interaction of GADD34 and eukaryotic Initiation Factor 2α is salubrinal.

14. The method according to claim 10 wherein the organism is a human.

15. A method for preventing a cancerous condition comprising administering a prophylactically effective combination of a proteasome inhibitor and a protein that inhibits Grp78 function to an organism in need.

16. The method according to claim 15 wherein the cancerous condition is multiple myeloma.

17. The method according to claim 15 wherein the proteasome inhibitor is selected from the group consisting of MG 132 and Velcade®.

18. The method according to claim 15 wherein the organism is a human.

19. A kit for treating or preventing a cancer comprising a therapeutically or prophylactically effective combination of a proteasomal inhibitor and a protein that interferes with the functional interaction of GADD34 and eukaryotic Initiation Factor 2α, along with a protocol guiding administration of the therapeutics or prophylactics.

20. A kit for treating or preventing a cancer comprising a therapeutically or prophylactically effective combination of a proteasomal inhibitor and a protein that inhibits Grp78 function, along with a protocol guiding administration of the therapeutics or prophylactics.
